(19) United States
(12) Patent Application Publication
(10) Pub. No.: US 2020/0348251 A1
(43) Pub. Date: Nov. 5, 2020

(54) GAS SENSOR MODULE FOR ELECTRONIC DEVICE AND ELECTRONIC DEVICE COMPRISING SAME

(71) Applicant: Samsung Electronics Co., Ltd., Gyeonggi-do (KR)

(72) Inventors: Min Ho PARK, Gyeonggi-do (KR); Seung Goo LEE, Gyeonggi-do (KR); Dong Uk KWAK, Gyeonggi-do (KR); Hyun Cheol PARK, Gyeonggi-do (KR); Sung Gun BAE, Gyeonggi-do (KR); Ik Joo BYUN, Gyeonggi-do (KR); Dae Ung JEONG, Gyeonggi-do (KR); Jeong Min PARK, Gyeonggi-do (KR)

(21) Appl. No.: 16/758,925

(22) PCT Filed: Oct. 25, 2018
(86) PCT No.: PCT/KR2018/012727

§ 371 Date: Apr. 24, 2020

(30) Foreign Application Priority Data
Nov. 1, 2017 (KR) 10-2017-0144810

(51) Int. Cl. G01N 27/12; G01N 33/00

(52) U.S. Cl. CPC G01N 27/125; G01N 33/0027

(57) ABSTRACT

Disclosed is a gas sensor module for an electronic device, the gas sensor module comprising: a gas sensor mounted on a circuit board and identifying gas introduced into a sensor unit; a sensor frame formed in a box shape with one open surface and having a hole communicating with the outside, the sensor frame covering the gas sensor to isolate the gas sensor from the outside; and a connection unit formed at an edge of the open surface of the sensor frame and coupled to the circuit board. Other embodiments are possible.

### TECHNICAL FIELD

This disclosure relates to a mounting structure of a gas sensor for improving the reliability of operation of a gas sensor in mounting and using the gas sensor in an electronic device.

### BACKGROUND ART

Recently, mobile communication terminals have been reduced in weight and size with the development of the hardware performance thereof, and have reached a mobile convergence stage in which the mobile communication terminals provide functions of other devices in addition to simple communication functions.

For example, a mobile communication terminal performs a function for measuring a simple health condition, and also performs a function for measuring the pollution level of surrounding air.

Development of technology for deriving more stable and accurate measurement results in these processes has been actively conducted.

### DISCLOSURE OF INVENTION

### Technical Problem

A gas sensor module for an electronic device according to an embodiment of the disclosure and an electronic device including the same are capable of spatially isolating a gas sensor from peripheral components and thus capable of blocking an effect of outgassing gas generated from the peripheral components.

### Solution to Problem

A gas sensor module for an electronic device according to an embodiment of the disclosure may include: a gas sensor mounted on a circuit board and configured to identify gas introduced into a sensor unit; a sensor frame having a shape of a box having one open surface and configured to cover the gas sensor so as to isolate the gas sensor from the outside, the sensor frame having a hole formed to communicate with the outside; and a connection portion formed at an edge of the open surface of the sensor frame and coupled with the circuit board.

An electronic device according to an embodiment of the disclosure may include: a housing; a circuit board embedded in the housing; a gas sensor mounted on the circuit board and configured to identify gas introduced into a sensor unit; a sensor frame having a shape of a box having one open surface and embedded in the housing, the sensor frame being configured to cover the gas sensor so as to form an isolation space in the housing and having a hole formed to communicate with the outside; and a connection portion formed at an edge of the open surface of the sensor frame and coupled with the circuit board.

### Advantageous Effects of Invention

In a gas sensor module for an electronic device according to an embodiment of the disclosure and an electronic device including the same, a gas sensor is spatially isolated from peripheral components. Thus, it is possible to block the effect of outgassing gas generated from the peripheral components, and to prevent damage caused by an external shock, and foreign matter.

### BRIEF DESCRIPTION OF DRAWINGS

FIG. 1A is a perspective view schematically illustrating the state in which a gas sensor module for an electronic device according to an embodiment of the disclosure is mounted, and

FIG. 1B is a view schematically illustrating a cross section of a portion in which a gas sensor module for an electronic device is mounted according to an embodiment of the disclosure;

FIG. 2A is a perspective view schematically illustrating the state in which a gas sensor module for an electronic device according to another embodiment of the disclosure is mounted, and FIG. 2B is a view schematically illustrating a cross section of a portion in which a gas sensor module for an electronic device is mounted according to an embodiment of the disclosure;

FIG. 3A is a perspective view schematically illustrating the state in which a gas sensor module for an electronic device according to another embodiment of the disclosure is mounted, and FIG. 3B is a view schematically illustrating a cross section of a portion of a gas sensor module for an electronic device according to another embodiment of the disclosure;

FIG. 4A is a perspective view schematically illustrating the state in which a gas sensor module for an electronic device according to another embodiment of the disclosure is mounted, and FIG. 4B is a view illustrating the state in which the position of the hole in the sensor frame is changed in the gas sensor module for an electronic device according to an embodiment of the disclosure; and

FIGS. 5A to 5C are views illustrating a connection portion of the gas sensor module for an electronic device according to an embodiment of the disclosure.

### MODE FOR THE INVENTION

Hereinafter, various embodiments of this document will be described with reference to the accompanying drawings. However, it should be understood that technology described in this document is not limited to a specific embodiment and includes various modifications, equivalents, and/or alternatives of an embodiment of this document. The same reference numbers are used throughout the drawings to refer to the same or like parts. In this document, an expression such as “A or B”, “at least one of A or/and B”, or “one or more of A or/and B” may include all possible combinations of together listed items. An expression such as “first” and “second” used in this document may indicate various constituent elements regardless of order and/or importance, is used for distinguishing a constituent element from another constituent element, and does not limit corresponding constituent elements. When it is described that a constituent element (e.g., a first constituent element) is “(operatively or communicatively) coupled with/to” or is “connected to” another constituent element (e.g., a second constituent element), it should be understood that the constituent element may be directly connected to the another constituent element or may be connected to the another constituent element through another constituent element (e.g., a third constituent element).

An expression “configured to” used in this document may be interchangeably used with, for example, “suitable for”, “having the capacity to”, “designed to”, “adapted to”, “made to”, or “capable of” according to a situation. A term “configured to” does not always mean “specifically designed to” in hardware. Alternatively, in any situation, an expression “device configured to” may mean that the device is “capable of” being configured together with another device or component.

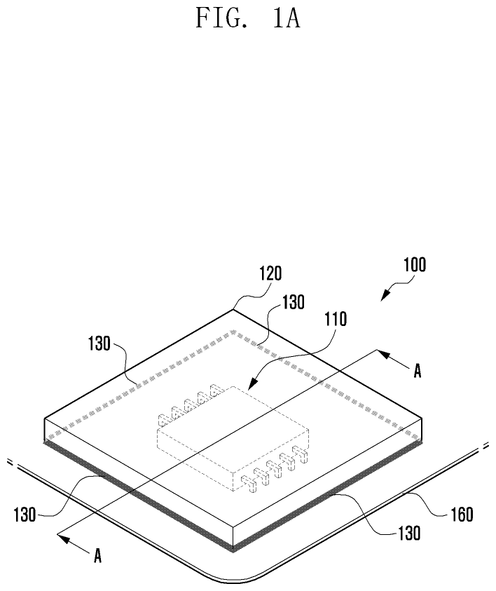

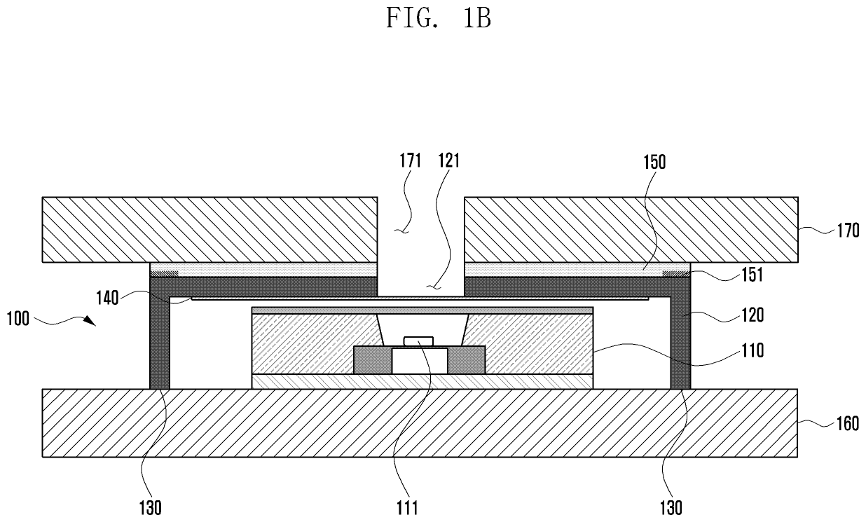

FIG. 1A is a perspective view schematically illustrating the state in which a gas sensor module 100 for an electronic device according to an embodiment of the disclosure is mounted, and may be a view schematically illustrating an arrangement relationship between a gas sensor 110, a sensor frame 120, the connection portion 130, or the like and a circuit board 160.

FIG. 1B is a view schematically illustrating a cross section of a portion in which the gas sensor module 100 for an electronic device according to an embodiment of the disclosure is mounted. FIG. 1B is a view taken along line A-A in FIG. 1A.

The gas sensor module 100 according to an embodiment of the disclosure may include a gas sensor 110, a sensor frame 120, a connection portion 130, a protective film 140, and a bumper 150. The sensor frame 120 may be the same as a component called a cover member 120.

The gas sensor 110 according to an embodiment of the disclosure may identify the concentration, type, or the like of the gas introduced into a sensor unit 111 inside the gas sensor 110. In order to identify the gas, the gas sensor 110 may use various measurement schemes such as a semiconductor-type scheme, an electrochemical-type scheme, a contact combustion-type scheme, and an optical-type scheme.

As the gas sensor 110 according to an embodiment of the disclosure, a semiconductor-type gas sensor may be used. The semiconductor-type gas sensor identifies an introduced gas using a scheme in which a metal oxide, layer provided in the sensor unit 111 comes into contact with the introduced gas, causing an oxidation-reduction reaction and the introduced gas is identified by measuring the changed resistance value of the metal oxide layer. The sensor unit 111 of the gas sensor may be opened to facilitate the introduction of the gas to be identified. However, the sensor unit 111 may be covered with a protective film 140 in order to prevent the introduction of foreign matter such as dust.

The gas sensor 110 may be mounted on the circuit board 160 of the electronic device using surface mount technology. In addition to the gas sensor 110, various components may be mounted on the circuit board 160 of the electronic device. Depending on the material of the components to be mounted, an outgassing phenomenon may occur. The outgassing phenomenon may mean a phenomenon in which gas that has been dissolved, trapped, frozen, or absorbed in a material constituting a component (e.g., a battery or a circuit board) is released.

In electronic devices, which are assemblies of a large number of components, the large number of components may be integrated in a narrow space due to but may be gradual reduction in weight and size thereof. Accordingly, the gas emitted due to the outgassing phenomenon may be introduced into the sensor unit 111 of the gas sensor 110. When the released gas is introduced into the gas sensor 110, it may affect the measured value of the gas sensor 110.

Accordingly, in the gas sensor module 100 for an electronic device according to an embodiment of the disclosure, a sensor frame 120 may be disposed so as to isolate the gas sensor 110 from the surrounding environment.

The sensor frame 120 may have a box shape having one open surface. In the drawings of this disclosure, a rectangular box-shaped sensor frame 120 is illustrated as a reference, but the sensor frame is not limited thereto, and may have any shape as long as the sensor frame 120 can cover the gas sensor 110 and shield the gas sensor 110 from the surrounding environment. For example, the sensor frame 120 may be cylindrical or may be formed in a dome shape.

The gas sensor 110 may pass through the open surface of the sensor frame 120 to be covered by the sensor frame 120, so that the gas sensor 110 can be isolated from the surrounding environment. In order to prevent the outgassing gas from being introduced into the sensor unit 111 of the gas sensor 110 by isolating the gas sensor from the surrounding environment, the airtightness of the connection portion 130, which is a connection portion between the sensor frame 120 and the circuit board 160, is very important.

Accordingly, in an embodiment of this disclosure, the sensor frame 120 may be fixed by soldering the edges of the open surface thereof to the circuit board 160. Through the soldering, it is possible to remove a gap, through which the outgassing gas generated from the electronic device is introduced, in the connection portion 130 between the circuit board 160 and the sensor frame 120.

Since it is necessary to isolate the gas sensor 110 from the surrounding environment and to ensure a passage through which a gas to be identified is to be introduced into the sensor unit 111, a hole may be formed in the sensor frame 120. The hole 121 in the sensor frame may also be referred to as a second opening 121. The position of the hole 121 in the sensor frame may be located to correspond to the position of the sensor unit 111 such that gas is easily supplied to the sensor unit 111 of the gas sensor 110.

Since it is necessary to block the inflow of a foreign substance such as moisture or dust in addition to the gas to be identified through the hole 121 in the sensor frame, a protective film 140 may be disposed so as to cover the hole 121 in the sensor frame.

The gas sensor module 100 for an electronic device according to an embodiment of this disclosure may be disposed inside the electronic device, that is, in the housing 170 of the electronic device. Accordingly, the outgassing gas generated from other components in the electronic device may flow through the gap between the housing 170 and the sensor frame 120 and through the hole 121 in the sensor frame.

In order to prevent this, the housing 170 of the electronic device and the sensor frame 120 may be in close contact with each other. However, in this case, a shock transmitted from the outside of the electronic device may be directly transmitted to the gas sensor module 100, and thus the gas sensor module may be damaged. Accordingly, the gas sensor module 100 for an electronic device according to an embodiment of the disclosure may include a bumper 150 formed of an elastic material and disposed between the housing 170 and the sensor frame 120 of the electronic device.

The bumper 150 may absorb the shock transmitted from the outside of the electronic device and block the transmission of the shock to the gas sensor module 100, thereby preventing damage to the gas sensor module 100, as well as preventing the outgassing gas from being introduced into the sensor unit 111 through the gap between the housing 170 and the sensor frame 120.

However, since the sensor frame 120 and the bumper 150 are components that are disposed adjacent to the sensor unit 111 of the gas sensor 110 and directly exposed, and may thus formed of a material that generates less outgassing gas. An outgassing degree may be expressed as a total mass loss (TLM) value, which is a value representing the amount of change in mass before and after processing for each material. The lower the TML value, the less the change in mass before and after processing of a material. A material having a lower TML value is a material that generates less outgassing gas.

It is necessary for the sensor frame 120 according to an embodiment of the disclosure to generate less outgassing gas as well as to serve to protect the gas sensor 110. Accordingly, the sensor frame 120 may be made of a metal material such as stainless steel, or a polycarbonate-based material, or a poly methyl methacrylate (PMMA)-based material.

It is necessary for the bumper 150 according to an embodiment of the disclosure to generate less outgassing gas as well as to have elasticity so as to absorb a shock transmitted from the outside of the electronic device and to fill the gap between the housing 170 and the sensor frame 120. Accordingly, the bumper 150 may be made of a material such as a polyethylene-based material.

The bumper 150 according to an embodiment of the disclosure may be coupled by being bonded to the sensor frame 120. The bumper 150 is disposed on a gas inflow path, and thus needs to be disposed so as not to cover the hole 121 in the sensor frame or the hole 171 in the housing. The hole 171 in the housing may also be referred to as a first opening 171.

When the position of the bumper 150 is not correctly fixed, the position of the bumper 150 may be shifted during the assembly process thereof and may interfere with the gas inflow path. Accordingly, since the bumper 150 according to an embodiment of the disclosure is fixed by being coupled to the sensor frame 120, it is possible to eliminate the risk that the position of the bumper 150 is displaced in the process of assembling the gas sensor module 100 inside the electronic device.

The bumper 150 and the sensor frame 120 may be coupled using an adhesive or adhesive tape 151. However, since the adhesive is highly likely to generate outgassing gas, the bumper 150 may be coupled in consideration of the bonded position so as to prevent the adhesive from being introduced into the sensor unit 111.

Referring to FIG. 1, the bumper 150 according to an embodiment of the disclosure may be coupled to a surface in which the hole 121 of the sensor frame is formed, and may be bonded along the peripheral portion thereof. By placing the adhesive to be spaced the most away from the hole 121 in the sensor frame through which the gas is introduced, it is possible to minimize the introduction of the outgassing gas.

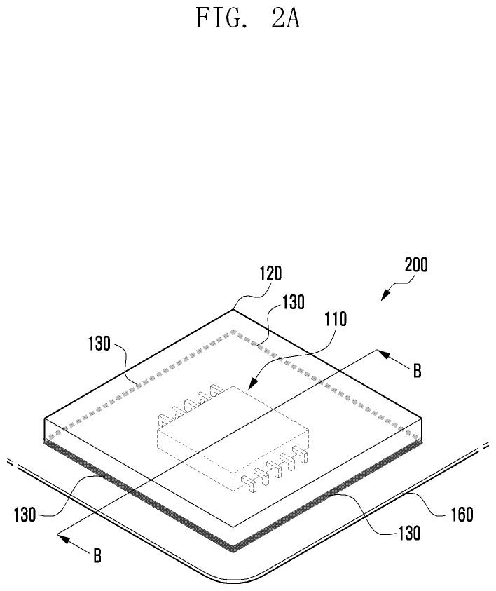

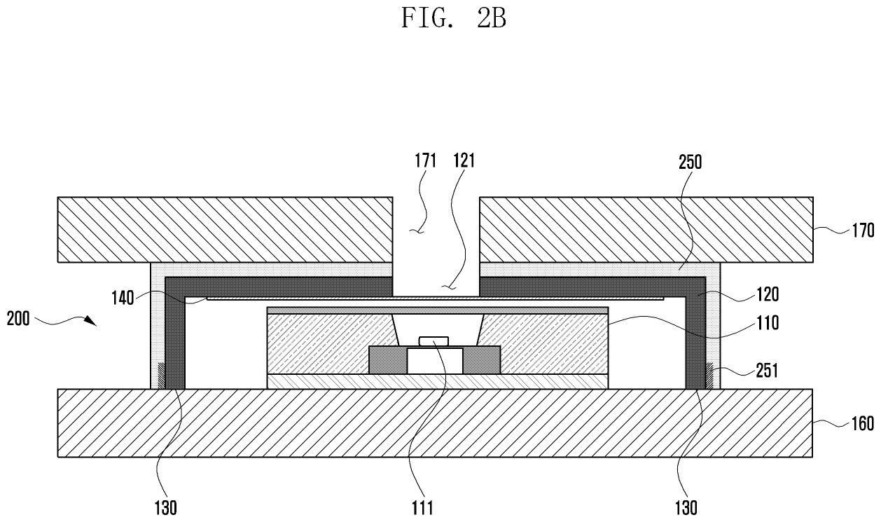

FIG. 2A is a perspective view schematically illustrating the state in which a gas sensor module 200 for an electronic device according to an embodiment of the disclosure is mounted. FIG. 2A may be a view schematically illustrating an arrangement relationship between the gas sensor 110, the sensor frame 120, the connection portion 130, or the like and the circuit board 160.

FIG. 2B is a view schematically illustrating a cross section of a portion in which the gas sensor module 200 for an electronic device according to an embodiment of the disclosure is mounted. FIG. 2B may be a view taken along line B-B in FIG. 2A.

In describing a gas sensor module 200 for an electronic device according to an embodiment of the disclosure, the same components as those in the embodiments of FIGS. 1A and 1B will be denoted by the same reference numerals, and a description thereof will be omitted. Hereinafter, a description will be made focusing on differences.

As illustrated in FIG. 2B, the gas sensor module 200 according to an embodiment of the disclosure may include a gas sensor 110, a sensor frame 120, a connection portion 130, a protective film 140, and a bumper 250.

Referring to FIG. 2B, the bumper 250 according to an embodiment of the disclosure may be formed to extend to the surface in which the hole 121 in the sensor frame is formed and to a surface adjacent thereto, and the extension end portion of the bumper 250 may be bonded to the sensor frame 120. Compared to the embodiment of FIG. 1B, the hole 121 in the sensor frame into which gas is introduced and the adhesive or adhesive tape 251 may be disposed so as to be further spaced apart from each other. Thus, the generated outgassing gas diffuses into the electronic device, thereby minimizing the introduction of the outgassing gas into the gas sensor 110.

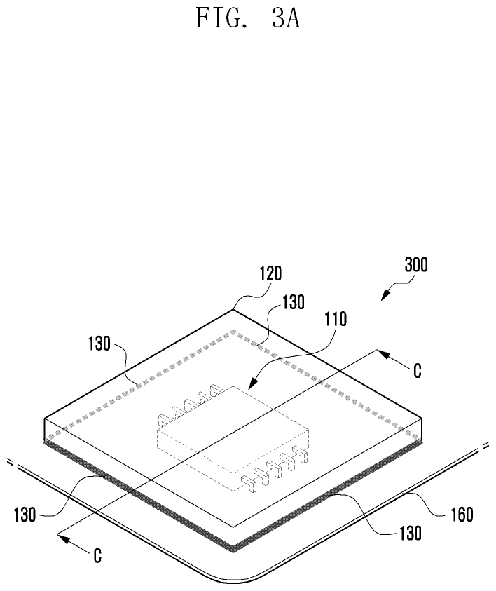

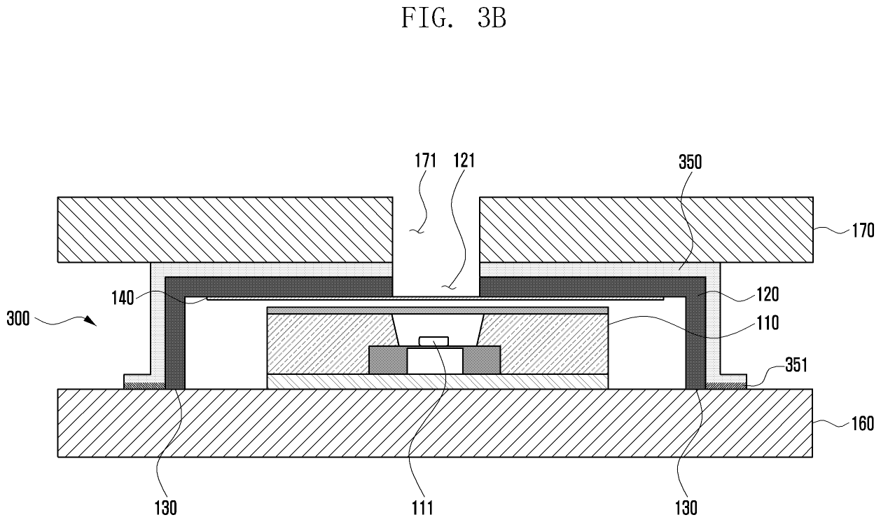

FIG. 3A is a perspective view schematically illustrating the state in which a gas sensor module 300 for an electronic device according to an embodiment of the disclosure is mounted. FIG. 3A may be a view schematically illustrating an arrangement relationship between the gas sensor 110, the sensor frame 120, the connection portion 130, or the like and the circuit board 160.

FIG. 3B is a view schematically illustrating a cross section of a portion in which the gas sensor module 300 for an electronic device according to an embodiment of the disclosure is mounted. FIG. 3B may be a view taken along line C-C in FIG. 3A.

In describing a gas sensor module 300 for an electronic device according to an embodiment of the disclosure, the same components as those in the embodiments of FIGS. 1A and 1B will be denoted by the same reference numerals, and a description thereof will be omitted. Hereinafter, a description will be made focusing on differences.

As illustrated in FIG. 3B, the gas sensor module 300 according to an embodiment of the disclosure may include a gas sensor 110, a sensor frame 120, a connection portion 130, a protective film 140, and a bumper 150.

Referring to FIG. 3B, the bumper 350 according to an embodiment of the disclosure may be formed to extend to the surface in which the hole 121 in the sensor frame is formed and to a surface adjacent thereto, and to further extend beyond the sensor frame 120. The extension end portion of the bumper 350 may be bonded to the sensor frame 160. Compared to the embodiment of FIG. 2B, the hole 121 in the sensor frame into which gas is introduced and the adhesive or adhesive tape 351 may be disposed so as to be further spaced apart from each other. Thus, the generated outgassing gas diffuses into the electronic device, thereby minimizing the introduction of the outgassing gas into the gas sensor 110.

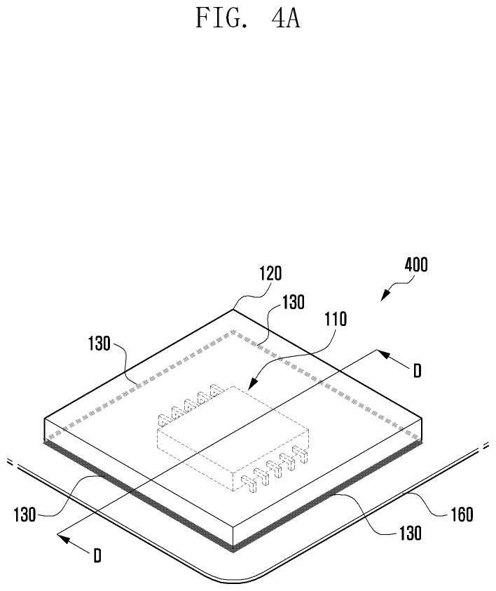

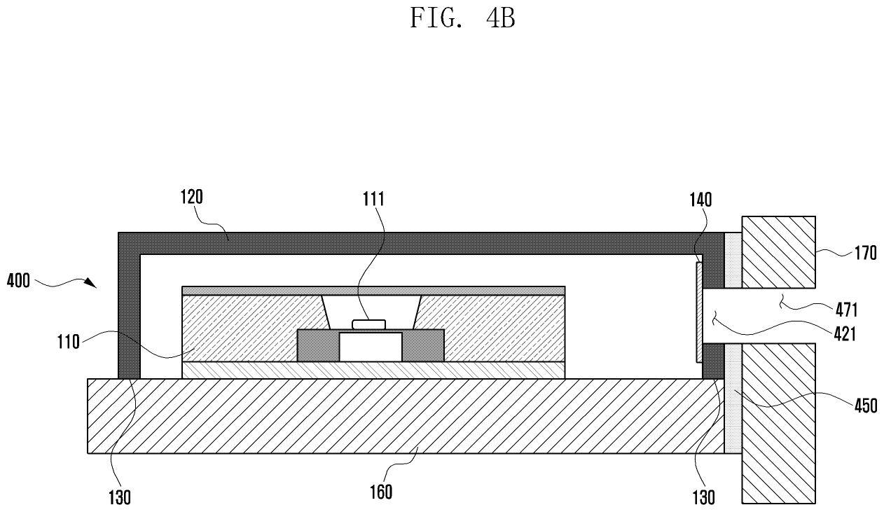

FIG. 4A is a perspective view schematically illustrating the state in which a gas sensor module 400 for an electronic device according to an embodiment of the disclosure is mounted. FIG. 4A may be a view schematically illustrating an arrangement relationship between the gas sensor 410, the sensor frame 420, the connection portion 430, or the like and the circuit board 460. FIG. 4B is a view illustrating the state in which the position of the hole 421 in the sensor frame is changed in the gas sensor module 400 for an electronic device according to an embodiment of the disclosure. FIG. 4B may be a view taken along line D-D in FIG. 4A. In describing a gas sensor module 400 for an electronic device according to an embodiment of the disclosure, the same components as those in the embodiments of FIGS. 1A and 1B will be denoted by the same reference numerals, and a description thereof will be omitted. Hereinafter, a description will be made focusing on differences.

The gas sensor module 400 according to an embodiment of the disclosure may include a gas sensor 110, a sensor frame 120, a connection portion 130, a protective film 140, and a bumper 450.

When the positions of a hole 471 formed in an electronic device housing 470 and a hole formed in the sensor frame 120 correspond to the position of the sensor unit 111 of the gas sensor 110, external gas may be smoothly introduced. However, when a material such as a sharp needle is inserted, the sensor unit 111 of the gas sensor 110 may be damaged.

Accordingly, this may be prevented in the gas sensor module 400 for an electronic device according to an embodiment of the disclosure, by disposing the hole 421 in the sensor frame to be out of alignment with the position of the sensor unit 111 of the gas sensor 110. For example, as illustrated in FIG. 4B, the hole 471 in the housing and the hole 421 in the sensor frame may be arranged to face the side face of the sensor unit 111.

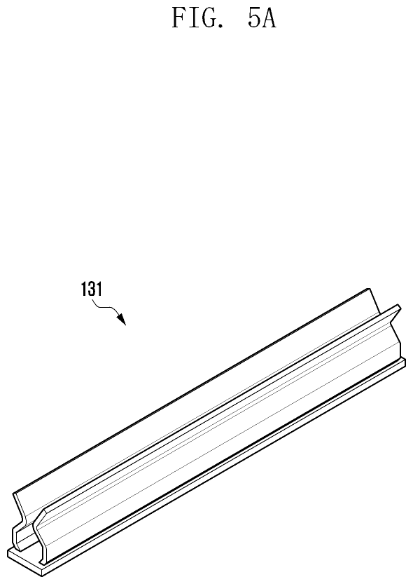

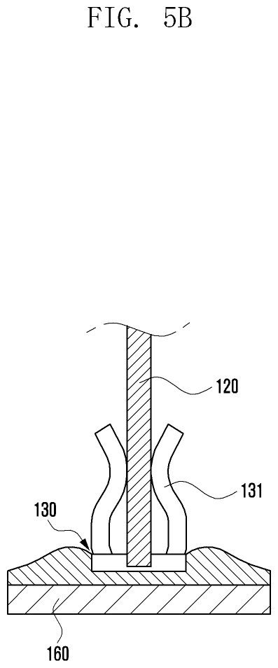

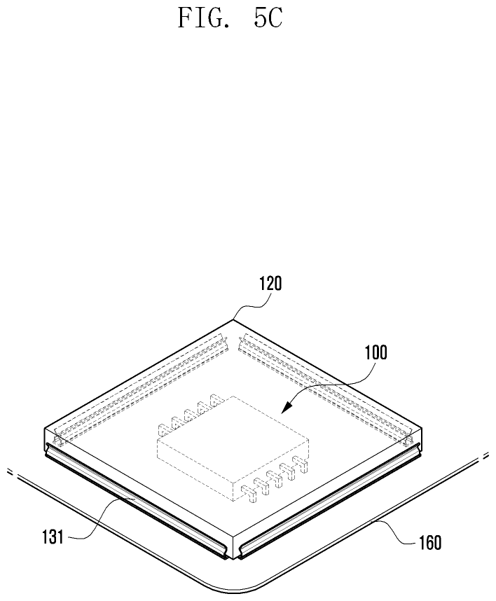

FIGS. 5A to 5C are views illustrating a connection portion (e.g., the connection portion 130 in FIG. 1) of the gas sensor module 100 for an electronic device according to an embodiment of the disclosure. FIG. 5A is a view illustrating a clip 131 according to an embodiment of the disclosure, FIG. 5B is a view illustrating a cross section when the sensor frame 120 is coupled with the clip 131 illustrated in FIG. 5A, and FIG. 5C is a view illustrating the state in which the sensor frame 120 is attached to the clip 131 mounted on the circuit board 160.

As described above, the airtightness of the portion, in which the sensor frame 120 is coupled to the circuit board 160, may be very important for blocking the introduction of outgassing gas. However, when direct soldering is performed, it takes time to assemble the sensor frame 120 through soldering, and may cause inconvenience when repairing or checking the gas sensor 110 later.

Accordingly, as illustrated in FIG. 5, the sensor frame 120 may be coupled to the circuit board 160 using the clip 131. Through this, since the coupling and separation of the sensor frame 120 are facilitated, repair and inspection of the gas sensor 110 may be facilitated, and assembly of the sensor frame 120 and the circuit board 160 may be facilitated.

A gas sensor module for an electronic device according to an embodiment of the disclosure may include: a gas sensor mounted on a circuit board and configured to identify gas introduced into a sensor unit; a sensor frame having a shape of a box having one open surface and configured to cover the gas sensor so as to isolate the gas sensor from the outside, the sensor frame having a hole formed to communicate with the outside; and a connection portion formed at an edge of the open surface of the sensor frame and coupled with the circuit board.

The gas sensor module may further include a protection film disposed to cover the hole in the sensor frame and configured to prevent dust or water from being introduced into the gas sensor through the hole.

The hole in the sensor frame may be located at a position corresponding to the position of the sensor unit when the gas sensor is covered.

The hole in the sensor frame may be located at a position out of alignment with the position of the sensor unit when the gas sensor is covered.

The gas sensor module may further include a bumper disposed on a surface, in which the hole in the sensor frame is formed, and formed of an elastic material.

The bumper may be bonded to the sensor frame along a portion of an outer periphery of the surface in which the hole in the sensor frame is formed.

The bumper may extend to a surface connected to the surface in which the hole in the sensor frame is formed, and the extension portion of the bumper may be bonded to the sensor frame.

The bumper may extend to a surface connected to the surface in which the hole in the sensor frame is formed, and the extension portion of the bumper may be bonded to the circuit board.

The connection portion may be provided in such a way that the sensor frame and the circuit board are directly soldered.

The connection portion may include a clip soldered to the circuit board, and an edge of the open surface of the sensor frame may be inserted into and fastened to the clip.

An electronic device according to an embodiment of the disclosure may include: a housing; a circuit board embedded in the housing; a gas sensor mounted on the circuit board and configured to identify gas introduced into a sensor unit; a sensor frame having a shape of a box having one open surface and embedded in the housing, the sensor frame being configured to cover the gas sensor so as to form an isolation space in the housing and having a hole formed to communicate with the outside; and a connection portion formed at an edge of the open surface of the sensor frame and coupled with the printed circuit board.

The electronic device may further include a protection film disposed to cover the hole in the sensor frame and configured to prevent dust or water from being introduced into the gas sensor through the hole.

The housing may have a hole formed to communicate with the outside, the hole in the housing and the hole in the sensor frame may be located at positions corresponding to a position of the sensor unit when the gas sensor is covered.

The housing may have a hole formed to communicate with the outside, and the hole in the housing and the hole in the sensor frame may be located at positions corresponding to each other and out of alignment with of a position of the sensor unit.

The electronic device may further include a bumper on a surface, in which the hole in the sensor frame is formed, and formed of an elastic material, the bumper being in contact with an inner surface of the housing.

The bumper may be bonded to the sensor frame along a portion of an outer periphery of the surface in which the hole in the sensor frame is formed.

The bumper may extend to a surface connected to the surface in which the hole in the sensor frame is formed, and the extension portion of the bumper may be bonded to the sensor frame.

The bumper may extend to a surface connected to the surface in which the hole in the sensor frame is formed, and the extension portion of the bumper may be bonded to the circuit board.

The connection portion may be of a type in which the sensor frame and the circuit board are directly soldered.

The connection portion may include a clip soldered to the circuit board, and an edge of the open surface of the sensor frame may be inserted into and fastened to the clip.

An electronic device according to an embodiment of the disclosure may include: a housing including a first opening; a circuit board disposed in an inner space of the housing; a gas sensor disposed on one surface of the circuit board and having a sensor unit disposed to face the first opening; and a cover member configured to house the gas sensor disposed on the one surface of the circuit board and having a second opening corresponding to the sensor unit of the received gas sensor.

An electronic device according to an embodiment of the disclosure may include: a housing including a first opening; a circuit board disposed in an inner space of the housing; a gas sensor disposed on one surface of the circuit board; and a cover member configured to house the gas sensor disposed on the one surface of the circuit board and having a second opening disposed to face the first opening.

An electronic device according to an embodiment of the disclosure may include: a housing including a first opening; a circuit board disposed in an inner space of the housing; a gas sensor disposed on one surface of the circuit board; and a cover member configured to house the gas sensor disposed on the one surface of the circuit board and having a second opening disposed to face the first opening. The cover member may be bonded to the circuit board using an adhesive member.

1. A gas sensor module for an electronic device, the gas sensor module comprising:
a gas sensor mounted on a printed circuit board and configured to identify gas introduced into a sensor unit;
a sensor frame having a shape of a box having one open surface and configured to cover the gas sensor so as to isolate the gas sensor from an outside, the sensor frame having a hole formed to communicate with the outside; and
a connection portion formed at an edge of the open surface of the sensor frame and coupled with the printed circuit board.

2. The gas sensor module of claim 1, further comprising:
a protection film disposed to cover the hole in the sensor frame and configured to prevent dust or water from being introduced into the gas sensor through the hole.

3. The gas sensor module of claim 1, wherein the hole in the sensor frame is located at a position corresponding to a position of the sensor unit when the gas sensor is covered.

4. The gas sensor module of claim 1, wherein the hole in the sensor frame is located at a position out of alignment with a position of the sensor unit when the gas sensor is covered.

5. The gas sensor module of claim 1, further comprising:
a bumper disposed on a surface, in which the hole in the sensor frame is formed, and formed of an elastic material.

6. An electronic device comprising:
a housing;
a printed circuit board embedded in the housing;
a gas sensor mounted on the printed circuit board and configured to identify gas introduced into a sensor unit;
a sensor frame having a shape of a box having one open surface and embedded in the housing, the sensor frame being configured to cover the gas sensor so as to form an isolation space in the housing and having a hole formed to communicate with an outside; and
a connection portion formed at an edge of the open surface of the sensor frame and coupled with the printed circuit board.

7. The electronic device of claim 6, further comprising:
a protection film disposed to cover the hole in the sensor frame and configured to prevent dust or water from being introduced into the gas sensor through the hole.

8. The electronic device of claim 6, wherein the housing has a hole formed to communicate with the outside, and
the hole in the housing and the hole in the sensor frame are located at positions corresponding to a position of the sensor unit when the gas sensor is covered.

9. The electronic device of claim 6, wherein the housing has a hole formed to communicate with the outside, and
the hole in the housing and the hole in the sensor frame are located at positions corresponding to each other and out of alignment with a position of the sensor unit.

10. The electronic device of claim 6, further comprising:
a bumper disposed on a surface, in which the hole in the sensor frame is formed, and formed of an elastic material, the bumper being in contact with an inner surface of the housing.

11. The electronic device of claim 10, wherein the bumper is bonded to the sensor frame along a portion of an outer periphery of the surface in which the hole in the sensor frame is formed.

12. The electronic device of claim 10, wherein the bumper extends to a surface connected to the surface in which the hole in the sensor frame is formed, and the extension portion of the bumper is bonded to the sensor frame.

13. The electronic device of claim 10, wherein the bumper extends to a surface connected to the surface in which the hole in the sensor frame is formed, and the extension portion of the bumper is bonded to the printed circuit board.

14. The electronic device of claim 6, wherein the connection portion is provided in such a way that the sensor frame and the printed circuit board are directly soldered.

15. The electronic device of claim 6, wherein the connection portion includes a clip soldered to the printed circuit board, and
an edge of the open surface of the sensor frame is inserted into and fastened to the clip.
